(19) United States
(12) Patent Application Publication
(10) Pub. No.: US 2018/0319889 A1
(43) Pub. Date: Nov. 8, 2018

(54) METHOD AND MEDICAMENT FOR TREATING AIRWAY AND/OR LUNG DISEASES

(71) Applicants: La Jolla Institute for Allergy & Immunology, La Jolla, CA (US); KYOWA HAKKO KIRIN CO., LTD, Tokyo (JP)

(72) Inventors: Michael CROFT, Escondido, CA (US); Rana HERRO, San Diego, CA (US); David MILLS, Seattle, WA (US); Rachel SOLOFF NUGENT, San Diego, CA (US); Monica LEUNG, San Diego, CA (US)

(73) Assignees: La Jolla Institute for Allergy & Immunology, La Jolla, CA (US); KYOWA HAKKO KIRIN CO., LTD, Tokyo (JP)

(21) Appl. No.: 15/772,909

(22) PCT Filed: Nov. 2, 2016
(86) PCT No.: PCT/JP2016/004806

§ 371 Date: May 2, 2018

Related U.S. Application Data
(60) Provisional application No. 62/249,532, filed on Nov. 2, 2015.

(51) Int. Cl. C07K 16/28; A61K 38/17; A61K 38/19; A61P 11/00; A61P 11/06

(52) U.S. Cl. CPC C07K 16/2878; A61K 38/1793; A61K 2039/505; A61P 11/00; A61P 11/06; A61K 38/191

(57) ABSTRACT

The present invention relates to a method and a medicament for airway and/or lung diseases such as moderate and/or severe asthma disease, chronic obstructive pulmonary disease (COPD), idiopathic pulmonary fibrosis (IPF), and systemic sclerosis. The medicament contains an agent which inhibits TL1A-DR3 interaction, and the method involves administering an effective amount of the agent to a subject to inhibit at least one symptom, feature or condition selected from hyperplasia of epithelial cells, epithelial metaplasia, hypertrophy of smooth muscle cells, and hyperproliferation of smooth muscle cells, production of extracellular matrix, and/or airway and/or lung tissue remodeling.

### TECHNICAL FIELD

The present invention relates to a method for the treatment of airway and/or lung diseases, as well as a medicament useful for the same. In particular, the invention provides a method comprising administering an effective amount of an agent which inhibits the interaction of the TNF family cytokine TL1A (TNFSF15) with the DR3 (TNFRSF25) receptor (hereinafter TL1A-DR3 or TL1A-DR3 interaction) to a subject or patient. The method also involves inhibiting TL1A-DR3 signal transduction. In addition, the present invention relates to a medicament or pharmaceutical composition useful for treating a subject with an airway and/or lung disease comprising an agent which inhibits TL1A-DR3 interaction, TL1A-DR3 signal transduction, or a combination thereof.

### BACKGROUND ART

All references cited herein are incorporated by reference in their entirety.

The airways and/or lungs of patients with airway and/or lung diseases such as, for example, asthma (moderate to severe), chronic obstructive pulmonary disease (COPD), idiopathic pulmonary fibrosis (IPF), and systemic sclerosis exhibit structural changes termed airway remodeling or lung tissue remodeling, primarily characterized by, but not limited to, smooth muscle hyperplasia and hypertrophy, and deposition of extracellular matrix proteins such as collagen, periostin, and fibronectin. These features are thought to be the primary cause of declining lung function in severe asthmatics (Non Patent Literatures 1 to 3; the disclosures of which are herein incorporated by reference in their entirety).

Molecules that contribute to these processes are then relevant for clinical intervention. In particular, receptors that are expressed on non-hematopoietic cells, especially structural cells in the lung, may be of great significance for blunting lung tissue remodeling associated with these diseases. Lung epithelial cells and lung smooth muscle cells can contribute directly to decline in lung function by expanding in size (hypertrophy), proliferating (hyperplasia), or producing extracellular matrix (ECM) proteins (e.g. collagen, fibronectin, periostin, others). TL1A is an inducible molecule, found membrane-bound or soluble, and can be made by T cells, dendritic cells, macrophages, and endothelial cells (Non Patent Literatures 4 to 6; the disclosures of which are herein incorporated by reference in their entirety).

TL1A acts through a single receptor DR3 (TNFRSF25). DR3 has been described to be expressed on lymphoid cells, predominantly T cells and innate lymphoid cells (ILC). TL1A has been associated with mild allergic acute lung inflammation as it can enhance cytokine production from Th2 cells and innate lymphoid type 2 cells (ILC2) that express DR3 (Non Patent Literatures 7 to 9; the disclosures of which are herein incorporated by reference in their entirety).

### CITATION LIST

### Non Patent Literature

NPL 1: Kumar et al, Pharmacol Ther. 2001; 91(2):93-104
NPL 2: Davies et al, J Allergy Clin Immunol. 2003; 111(2):215-25
NPL 3: Pascual et al, J Allergy Clin Immunol. 2005; 116(3):477-86
NPL 4: Croft et al, Nat Rev Immunol. 2009; 9(4):271-85
NPL 5: Croft et al., Trends Immunol. 2012; 33(3):144-52
NPL 6: Croft et al., Nat Rev Drug Discov. 2013; 12(2):147-68
NPL 7: Meylan et al, Immunity. 2008; 29(1):79-89
NPL 8: Meylan et al, Mucosal Immunol. 2014; 7(4):958-68
NPL 9: Yu et al, Mucosal immunology. 2013; 7(3):730-40

### SUMMARY OF INVENTION

The present invention provides a method and a medicament or pharmaceutical composition for preventing, alleviating, and/or treating airway and/or lung diseases including, but not limited to, moderate and/or severe asthma disease, chronic obstructive pulmonary disease (COPD), idiopathic pulmonary fibrosis (IPF), and systemic sclerosis. The method of the present invention comprises administering an effective amount of at least one agent which inhibits TL1A-DR3 interaction, TL1A signal transduction, or a combination thereof, in a subject with an airway and/or lung diseases.

In one embodiment, the method comprises administering an effective amount of at least one agent which inhibits TL1A-DR3 interaction, TL1A signal transduction, or a combination thereof, to prevent, reduce or inhibit at least one symptom, feature or condition, including but not limited to, hyperplasia of epithelial cells, epithelial metaplasia, hypertrophy of smooth muscle cells, hyperproliferation of smooth muscle cells, production of extracellular matrix, and airway and/or lung tissue remodeling in a subject with airway and/or lung diseases.

In one embodiment, the airway or lung disease is moderate and/or severe asthma disease, chronic obstructive pulmonary disease (COPD), idiopathic pulmonary fibrosis (IPF), or systemic sclerosis.

In a preferred embodiment, the moderate and/or severe asthma may be neutrophilic asthma or eosinophilic asthma.

In another preferred embodiment, the airway or lung disease is idiopathic pulmonary fibrosis.

In another embodiment, the lung or airway disease involves infiltration of at least one cell selected from an eosinophil and neutrophil.

In yet another embodiment, the subject has airway or lung tissue remodeling caused by the lung disease or airway disease. The airway or lung tissue remodeling is prevented, reduced, or inhibited by the inhibition of TL1A-DR3 interaction, TL1A-DR3 signal transduction or a combination thereof. The airway or lung tissue remodeling is characterized by at least one symptom, feature or condition selected from the group consisting of hyperplasia of epithelial cells, epithelial metaplasia, hypertrophy of smooth muscle cells, hyperproliferation of smooth muscle cells, and production of extracellular matrix.

The present invention also provides a method of preventing, reducing, and/or inhibiting airway or lung tissue remodeling comprising administering to a subject with lung disease or airway disease.

In one embodiment, the airway or lung tissue remodeling is characterized by extracellular matrix protein production.

The present invention provides a medicament for preventing, alleviating and/or treating a lung disease or airway disease comprising at least one agent which inhibits TL1A-DR3 interaction, TL1A-DR3 signal transduction or a combination thereof.

In one embodiment, at least one agent in the medicament inhibits at least one symptom, feature or condition caused by the lung disease or airway disease selected from the group consisting of hyperplasia of epithelial cells, epithelial metaplasia, hypertrophy of smooth muscle cells, hyperproliferation of smooth muscle cells, production of extracellular matrix, and airway and/or lung tissue remodeling.

In another embodiment, the medicament is useful for lung or airway disease that is moderate and/or severe asthma, chronic obstructive pulmonary disease, idiopathic pulmonary fibrosis or systemic sclerosis.

In the methods and medicaments of the present invention, at least one agent is a small molecule, an antibody and/or a decoy molecule.

In the methods and medicaments of the present invention, the antibody is an anti-TL1A antibody or an anti-DR3 antibody.

In the methods and medicaments of the present invention, the decoy molecule is a decoy TL1A ligand, a decoy DR3 receptor, or decoy receptor 3 (DcR3).

In a preferred embodiment of the methods and medicaments of the present invention, the decoy molecule is selected from the group consisting of a TL1A conjugated with a Fc region of immunoglobulin, a DR3 conjugated with a Fc region of immunoglobulin, and a decoy receptor 3 (DcR3) conjugated with a Fc region of immunoglobulin.

In another preferred embodiment of the methods and medicaments of the present invention, the anti-DR3 antibody is an anti-DR3 monoclonal antibody selected from the group consisting of:
an antibody comprising CDR1 to CDR3 of VH of SEQ ID NOs:2-4 and CDR1 to CDR3 of VL of SEQ ID NOs:6-8;
an antibody comprising CDR1 to CDR3 of VH of SEQ ID NOs:10-12 and CDR1 to CDR3 of VL of SEQ ID NOs:14-16;
an antibody comprising CDR1 to CDR3 of VH of SEQ ID NOs:2, 3, 21 and CDR1 to CDR3 of VL of SEQ ID NOs:6-8;
an antibody comprising CDR1 to CDR3 of VH of SEQ ID NOs:2, 3, 21 and CDR1 to CDR3 of VL of SEQ ID NOs:6, 22, 8;
an antibody comprising CDR1 to CDR3 of VH of SEQ ID NOs: 2, 3, 21 and CDR1 to CDR3 of VL of SEQ ID NOs:6, 23, 8;
an antibody comprising CDR1 to CDR3 of VH of SEQ ID NOs: 2, 3, 21 and CDR1 to CDR3 of VL of SEQ ID NOs:6, 24, 8;
an antibody comprising CDR1 to CDR3 of VH of SEQ ID NOs: 2-4 and CDR1 to CDR3 of VL of SEQ ID NOs: 6, 22, 8;
an antibody comprising CDR1 to CDR3 of VH of SEQ ID NOs:2-4 and CDR1 to CDR3 of VL of SEQ ID NOs: 6, 23, 8;
an antibody comprising CDR1 to CDR3 of VH of SEQ ID NOs:2-4 and CDR1 to CDR3 of VL of SEQ ID NOs: 6, 24, 8;
an antibody comprising VH of SEQ ID NO:1 and VL of SEQ ID NO:5;
an antibody comprising VH of SEQ ID NO:9 and VL of SEQ ID NO:13;
an antibody comprising VH of SEQ ID NO:1 and VL of SEQ ID NO:18;
an antibody comprising VH of SEQ ID NO:1 and VL of SEQ ID NO:19;
an antibody comprising VH of SEQ ID NO:1 and VL of SEQ ID NO:20;
an antibody comprising VH of SEQ ID NO:17 and VL of SEQ ID NO:18;
an antibody comprising VH of SEQ ID NO:17 and VL of SEQ ID NO:19; and an antibody comprising VH of SEQ ID NO:17 and VL of SEQ ID NO:20.

### BRIEF DESCRIPTION OF DRAWINGS

FIG. 1 shows that the receptor for TL1A, DR3, is expressed on human lung structural cells linked to tissue remodeling. Primary normal human airway smooth muscle cells (NHASM/BSMC) (FIG. 1A), purchased from Lonza Group, Ltd., and a human bronchial epithelial cell line (BEAS2-B) (FIG. 1B) were stained for DR3 with an anti-DR3 antibody. Isotype control is shown by dotted line. Data reveals that DR3 is expressed on structural cells that contribute to lung tissue remodeling.

FIG. 2 shows TL1A promotes tissue remodeling in human bronchial epithelial cells. FIG. 2A shows the mRNA expression levels of periostin relative to GAPDH and FIG. 2B shows the mRNA expression levels of alpha smooth muscle actin relative to GAPDH after stimulation with TL1A.

FIG. 3 shows that TL1A promotes tissue remodeling in airway smooth muscle cells. Airway smooth muscle cells are stimulated by TL1A to express mRNA of the extracellular matrix proteins collagen (FIG. 3A) and fibronectin (FIG. 3B) relative to GAPDH.

FIG. 4 shows that TL1A directly promotes lung tissue remodeling in wild-type mice. Quantitation of collagen deposition (area of trichrome staining) (FIG. 4A) and thickness of peribronchial smooth muscle (FIG. 4B) are shown.

FIG. 5 shows that TL1A directly promotes lung tissue remodeling in mice lacking T cells and innate lymphoid cells. Quantitation of collagen deposition (area of trichrome staining) (FIGS. 5A and 5C) and thickness of peribronchial smooth muscle (FIGS. 5B and 5D) are shown.

FIG. 6 shows that a genetic deficiency in DR3 reduces lung tissue remodeling driven by allergen. Accumulation of collagen (trichrome area) (FIG. 6A) and change in peribronchial smooth muscle thickness compared to controls (FIG. 6B) are shown.

FIG. 7 shows that therapeutic blocking of TL1A-DR3 interactions reduces lung tissue remodeling driven by allergen. Accumulation of collagen (trichrome area) (FIG. 7A) and change in peribronchial smooth muscle thickness compared to controls (FIG. 7B) are shown.

FIG. 8 shows that blocking of TL1A-DR3 interactions reduces lung tissue remodeling driven by bleomycin. Accumulation of collagen (trichrome area) (FIG. 8A) and peribronchial smooth muscle thickness compared to controls (FIG. 8B) are shown.

FIG. 9 shows that multiple inflammatory stimulants induce TL1A expression in neutrophils from healthy donors.

FIG. 10 shows that multiple inflammatory stimulants induce TL1A expression in eosinophils from healthy donors.

### DESCRIPTION OF EMBODIMENTS

The present invention provides a method and a medicament or pharmaceutical composition for preventing, alleviating and/or treating airway or lung disease.

The method of the present invention involves administering to a subject in need thereof, at least one agent which inhibits TL1A-DR3 interaction, TL1A-DR3 signal transduction, or a combination thereof. In particular, the airway and/or lung disease is moderate and/or severe asthma disease, chronic obstructive pulmonary disease (COPD), idiopathic pulmonary fibrosis (IPF), or systemic sclerosis.

The method of the present invention involves administering to a subject with airway or lung disease, at least one agent which inhibits TL1A-DR3 interaction, TL1A-DR3 signal transduction, or a combination thereof, to prevent, reduce, or inhibit at least one symptom, feature or condition that is selected from hyperplasia of epithelial cells, epithelial metaplasia, hypertrophy of smooth muscle cells, hyperproliferation of smooth muscle cells, extracellular matrix production, migration of neutrophils, invasion of neutrophils, migration of eosinophils, invasion of eosinophils, and airway and/or lung tissue remodeling.

The method of the present invention also involves administering to a subject with lung or airway disease, at least one agent which inhibits TL1A-DR3 interaction, TL1A-DR3 signal transduction, or a combination thereof, to prevent, reduce, or inhibit at least one symptom or condition that is characteristic of airway or lung tissue remodeling. The at least one symptom, feature or condition is selected from hyperplasia of epithelial cells, epithelial metaplasia, hypertrophy of smooth muscle cells, hyperproliferation of smooth muscle cells, extracellular matrix production, migration of neutrophils, invasion of neutrophils, migration of eosinophils, and invasion of eosinophils. In particular, the method of the present invention involves administering to a subject with lung or airway disease, at least one agent which inhibits TL1A-DR3 interaction, TL1A-DR3 signal transduction, or a combination thereof, to prevent, reduce, or inhibit extracellular matrix production.

The medicament of the present invention contains at least one agent which inhibits TL1A-DR3 interaction, TL1A-DR3 signal transduction, or a combination thereof. The medicament may be included in a pharmaceutical composition with a pharmaceutically acceptable carrier.

The pharmaceutical composition of the present invention contains at least one agent which inhibits TL1A-DR3 interaction, TL1A-DR3 signal transduction, or a combination thereof, and a pharmaceutically acceptable carrier.

The medicament or pharmaceutical composition of the present invention is useful for preventing, alleviating, and/or treating a subject with airway or lung disease. The medicament or pharmaceutical composition is also useful for treating airway or lung disease that is caused by airway or lung tissue remodeling.

The medicament or pharmaceutical composition of the present invention is also useful for preventing, reducing, or inhibiting at least one symptom feature, or condition selected from the group consisting of, but is not limited to, hyperplasia of epithelial cells, epithelial metaplasia, hypertrophy of smooth muscle cells, hyperproliferation of smooth muscle cells, production of extracellular matrix, airway and/or lung tissue remodeling, migration of neutrophils, invasion of neutrophils, migration of eosinophils, and invasion of eosinophils. In particular, the medicament or pharmaceutical composition of the present invention is useful for preventing, reducing, or inhibiting extracellular matrix production and/or airway and/or lung tissue remodeling.

Unless otherwise defined, the scientific and technical terms used herein have the meaning as commonly understood by one of ordinary skill in the art. The present invention is not limited to the methods, medicament or pharmaceutical compositions described herein. It is understood that one of ordinary skill in the art can modify the disclosed methods and medicaments or pharmaceutical compositions, and embodiments thereof to attain alternative or variant embodiments with alternative or variant usages, all of which are covered by the present application and can be within the scope of the application.

In the present invention, subjects with airway and/or lung disease may have “airway remodeling and/or lung tissue remodeling” or “airway and/or lung tissue remodeling” which may be prevented, reduced or inhibited by inhibition or blockage of TL1A-DR3 interaction, TL1A signal transduction, or a combination thereof. The airway and/or lung tissue remodeling may be characterized by at least one symptom, feature or condition, including, but not limited to, smooth muscle hyperplasia and hypertrophy, and deposition or production of extracellular matrix proteins such as collagen, periostin, and fibronectin. Alternatively, subjects with airway and/or lung disease may have at least one symptom, feature or condition, including, but not limited to, hyperplasia of epithelial cells, epithelial metaplasia, hypertrophy of smooth muscle cells, hyperproliferation of smooth muscle cells, production of extracellular matrix, and airway and/or lung tissue remodeling; which can be prevented, reduced or inhibited by inhibition or blockage of TLA-DR3 interaction, TL1A signal transduction, or a combination thereof.

The term “inhibit,” “inhibiting,” or “inhibition” means, but is not limited to, to block, impede, hinder, prevent, or slow down an activity. For instance, inhibition of TL1A-DR3 interaction may include, but is not limited to, the binding of a small molecule, antibody and/or a decoy molecule to TL1A or DR3, or both, to block, impede, hinder, or prevent the interaction between TL1A and the DR3 receptor. Likewise, inhibition of TL1A-DR3 signal transduction may include, for example, blocking, impeding, hindering, preventing, or slowing down of the function of any component involved in the TL1A-DR3 signal transduction pathway.

The term “alleviate” or “alleviation” means to reduce, relieve, mitigate, or attenuate. For instance, the inhibition of TL1A-DR3 interaction may include alleviating at least one symptom, feature or condition in a subject with lung disease or airway disease, including, but not limited to, hyperplasia of epithelial cells, epithelial metaplasia, hypertrophy of smooth muscle cells, hyperproliferation (such as hyperplasia) of smooth muscle cells, extracellular matrix protein production, inflammation and airway and/or lung tissue remodeling.

The term “measurable” or “measurably” means capable of being detected, detectable or discernible, and may represent a noticeable, observable, perceivable or visible difference or lack of difference. For instance, the inhibition of TL1A-DR3 interaction, TL1A-DR3 signal transduction, or a combination thereof, by an agent of the present invention in a subject with a lung or airway disease, may be detectable or observable relative to a control subject without the disease or relative to a subjected with a similar airway and/or lung disease who is not administered the agent. Also, the administration of an agent of the present invention to a subject with an airway and/or lung disease can provide for an observable, perceivable or visible difference attributable to prevention, alleviation, and/or treatment of an airway and/or lung disease, or at least one symptom, feature or condition associated with an airway and/or lung disease, relative to a subject with a similar airway and/or lung disease who is not administered the agent. Alternatively, the administration of at least one agent of the present invention may provide for a lack of observable, perceivable or visible difference between a subject with an airway and/or lung disease relative to a control subject without such a disease.

A “medicament” may mean an agent or a substance as described herein used to medically alleviate, prevent, and/or treat an airway and/or lung disease. In one embodiment, a medicament of the present invention may be included in a pharmaceutical composition. In another embodiment, the invention provides a pharmaceutical composition comprising a medicament of the invention in admixture with a carrier. The medicament of the present invention, may be formulated as pharmaceutical compositions prepared for storage or administration, and which comprise a therapeutically effective amount of medicament of the present invention, in a pharmaceutically acceptable carrier.

A pharmaceutical composition may be formulated in the form of a pill, granule, spray, liquid, etc., using conventional preparation methods. The pharmaceutically acceptable carriers used are not particularly limited and may include carriers that are used standardly by persons of skill in the art.

Routes of administration are not particularly limited, and may include, but are not limited to, intravenous administration, oral administration, administration via inhalation and topical administration. One of ordinary skill in the art will be able to determine the route of administration that is best suited for the subject in need depending on the symptoms and disease conditions exhibited.

As used herein, “an effective amount” or “a therapeutically effective amount” is one which reduces at least one symptom or feature of a given airway and/or lung disease condition or pathology, and preferably which normalizes physiological responses in an individual with the disease condition or pathology. Reduction of symptoms or normalization of physiological responses can be determined using methods routine in the art and may vary with a given condition or pathology. In one aspect, a “therapeutically effective amount” of at least one agent or medicament of the invention or a pharmaceutical composition comprising at least one agent or medicament of the invention is an amount which restores a measurable physiological parameter to substantially the same value (preferably to within 30%, more preferably to within 20%, and still more preferably, to within 10% of the value) of the parameter in an individual without the disease condition or pathology.

As is apparent to one skilled in the art, an “effective amount” or a “therapeutically effective amount” of at least one agent or medicament of the present invention, or a pharmaceutical composition of the present invention, will vary depending upon the age, weight and mammalian species treated, the particular compounds employed, the particular mode of administration and the desired effects and the therapeutic indication. Because these factors and their relationship to determining this amount are well known, the determination of an effective dosage level or therapeutically effective dosage levels, the amount necessary to achieve the desired result of alleviating, preventing and/or treating the lung or airway diseases described herein, will be within the skill of the skilled person.

For instance, an “effective amount” or a “therapeutically effective amount” of at least one agent or medicament of the present invention, or a pharmaceutical composition of the present invention, will depend on the route of administration, the type of mammal being treated, and the physical characteristics of the specific mammal under consideration. These factors and their relationship to determining this amount are well known to skilled practitioners in the medical arts. This amount and the method of administration can be tailored to achieve optimal efficacy so as to deliver the agent, medicament, or pharmaceutical composition to the lung and/or airway, but will depend on such factors as weight, diet, concurrent medication and other factors, well known to those skilled in the medical arts.

In one embodiment, an “effective amount” may be the amount associated with the alleviation, prevention, blocking and/or inhibition of airway and/or lung tissue remodeling and/or airway inflammation. In another, embodiment, an “effective amount” may be the amount associated with the alleviation, prevention, blocking and/or inhibition of at least one symptom, feature or disease condition exhibited by a subject with an airway and/or lung disease, that includes, but is not limited to hyperplasia of epithelial cells, epithelial metaplasia, hypertrophy of smooth muscle cells, hyperproliferation (hyperplasia) of smooth muscle cells, extracellular matrix protein production and airway and/or lung tissue remodeling.

According to one aspect of the present invention, a subject with an airway and/or lung disease may exhibit airway and/or lung tissue remodeling and/or airway inflammation. In another aspect of the invention, a subject with airway and/or lung disease may have at least one symptom, feature or condition including, but not limited to, hyperplasia of epithelial cells, epithelial metaplasia, hypertrophy of smooth muscle cells, hyperproliferation (such as hyperplasia) of smooth muscle cells, extracellular matrix protein production, airway and/or lung tissue remodeling, migration of neutrophils, invasion of neutrophils, migration of eosinophils, and invasion of eosinophils. By inhibition or blockage of TLA-DR3 interaction, TL1A signal transduction, or a combination thereof, a medicament or pharmaceutical composition of the present invention treats, prevents or reduces the exacerbation of the lung and/or airway disease, in particular, moderate and/or severe asthma (such as neutrophilic asthma or eosinophilic asthma), COPD, IPF, or systemic sclerosis.

Accordingly, a subject with lung or airway disease of the present invention may be described to exhibit airway and/or lung tissue remodeling. Alternatively, subject with lung or airway disease of the present invention may also be described to have at least one symptom, feature or condition selected from the group consisting of hyperplasia of epithelial cells, epithelial metaplasia, hypertrophy of smooth muscle cells, hyperproliferation (such as hyperplasia) of smooth muscle cells, extracellular matrix protein production, airway and/or lung tissue remodeling, migration of neutrophils, invasion of neutrophils, migration of eosinophils, and invasion of eosinophils.

An airway and/or lung disease may include, but is not limited to, lung disease affecting the airways, lung disease affecting the air sacs (alveoli), lung disease affecting the interstitium, lung disease affecting blood vessels, lung disease affecting the pleura, and lung diseases affecting the chest wall.

Preferably, the lung and/or airway disease is idiopathic pulmonary fibrosis (IPF), neutrophilic asthma, eosinophilic asthma, systemic sclerosis and steroid resistant forms thereof.

The term “antibody” includes, but is not limited to, polyclonal antibodies, multiclonal antibodies, monoclonal antibodies, chimeric antibodies, humanized and primatized antibodies, human antibodies, recombinantly produced antibodies, intrabodies, multispecific antibodies, bispecific antibodies, monovalent antibodies, multivalent antibodies, anti-idiotypic antibodies, synthetic antibodies, including mutations and variants thereof; antibody fragments such as Fab fragments, F(ab′) fragments, single-chain FvFcs, single-chain Fvs; and derivatives thereof including Fc fusions and other modifications, and any other immunologically active molecule so long as they exhibit the desired biological activity (i.e., antigen association or binding). Moreover, the term further includes all classes of antibodies (i.e. IgA, IgD, IgE, IgG, and IgM) and all isotypes (i.e., IgG1, IgG2, IgG3, IgG4, IgA1, and IgA2), as well as variations thereof unless otherwise dictated by context.

The present invention provides a method and a medicament of treating moderate and/or severe asthma disease, chronic obstructive pulmonary disease (COPD), idiopathic pulmonary fibrosis (IPF), or systemic sclerosis comprising administering an effective amount of at least one agent which inhibits TL1A-DR3 interaction in a subject, wherein the subject has at least one symptom, feature or condition selected from hyperplasia of epithelial cells, epithelial metaplasia, hypertrophy of smooth muscle cells, hyperproliferation of smooth muscle cells, production of extracelluar matrix, and airway and/or lung tissue remodeling. The present invention also provides a method and a medicament of treating moderate and/or severe asthma disease, chronic obstructive pulmonary disease (COPD), systemic sclerosis or idiopathic pulmonary fibrosis (IPF) comprising administering an effective amount of an agent which inhibits TL1A-DR3 interaction into a subject to prevent, reduce or inhibit at least one symptom, feature or condition selected from hyperplasia of epithelial cells, epithelial metaplasia, hypertrophy of smooth muscle cells, hyperproliferation of smooth muscle cells, production of extracelluar matrix and/or airway and/or lung tissue remodeling.

In a preferable embodiment, the present invention includes a method and a medicament for alleviating, preventing and/or treating moderate and/or severe asthma disease, COPD, systemic sclerosis or IPF comprising administering an effective amount of an agent which inhibits TL1A-DR3 interaction in a subject, wherein the subject exhibits airway and/or lung tissue remodeling. In another embodiment, the present invention includes a method and a medicament or pharmaceutical composition for alleviating, preventing and/or treating moderate and/or severe asthma disease, COPD, systemic sclerosis or IPF comprising administering an effective amount of at least one agent which inhibits TL1A-DR3 interaction, TL1A-DR3 signal transduction, or a combination thereof, in a subject, wherein the subject exhibits airway or lung tissue remodeling characterized by at least one symptom, feature or condition selected from hyperplasia of epithelial cells, epithelial metaplasia, hypertrophy of smooth muscle cells, and hyperproliferation of smooth muscle cells.

In another preferable embodiment, the present invention includes a method and a medicament or pharmaceutical composition for alleviating, preventing, and/or treating moderate and/or severe asthma disease, chronic obstructive pulmonary disease (COPD), systemic sclerosis or idiopathic pulmonary fibrosis (IPF) comprising administering an effective amount of at least one agent which inhibits TL1A-DR3 interaction, TL1A-DR3 signal transduction, or a combination thereof, to a subject, to inhibit at least one symptom, feature or condition associated with airway and/or lung tissue remodeling selected from hyperplasia of epithelial cells, epithelial metaplasia, hypertrophy of smooth muscle cells, hyperproliferation of smooth muscle cells, and production of extracellular matrix.

In one embodiment, the method and the medicament or pharmaceutical composition of the present invention prevents, alleviates, and/or treats moderate and/or severe asthma disease, COPD, systemic sclerosis or IPF which involves infiltration of at least one cell selected from an eosinophil and a neutrophil. In one preferable embodiment, the method and the medicament or pharmaceutical composition of the invention, prevents, alleviates and/or treats airway inflammation and/or airway or lung tissue remodeling that is caused by moderate and/or severe asthma disease, COPD, systemic sclerosis or IPF which involves the infiltration of mainly/dominantly neutrophils or eosinophils. In the most preferable embodiment, the method and the medicament or pharmaceutical composition of the present invention prevents, alleviates and/or treats moderate and/or severe neutrophilic asthma, eosinophilic asthma or IPF.

Infiltration of neutrophils or eosinophils refers to the movement of neutrophils or eosinophils into the tissues of airway and/or lung.

In the present invention, neutrophilic asthma means asthma in which neutrophils are mainly/dominantly infiltrated into tissues of airway and/or lung compared to any other immune cells such as a Th2 cell, eosinophil, or basophil. The neutrophilic asthma is normally diagnosed, when neutrophils are detected in an expectoration from a patient.

In the present invention, eosinophilic asthma means asthma in which eosinophils are mainly/dominantly infiltrated into tissues of airway and/or lung compared to any other immune cells such as a Th2 cell, neutrophil, or basophil. The eosinophilic asthma is normally diagnosed, when eosinophils are detected in an expectoration from a patient. In one preferable embodiment, the method and the medicament of the present invention treats moderate and/or severe asthma or IPF which is resistant to steroid treatment.

Steroid treatment means a treatment comprising an adrenocorticosteroidal hormone, including but not limited to, prednisolone, methylprednisolone, beclomethasone, deoxycorticosterone, dexamethasone, hydrocortisone, and cortisone, which are administered to prevent inflammatory reactions.

In one other embodiment, the method and the medicament or pharmaceutical composition of the present invention can be used to prevent, alleviate, and/or treat a moderate and/or severe asthma disease, COPD, systemic sclerosis or IPF accompanied by or comprising at least one symptom, feature or condition selected from hyperplasia of epithelial cells, epithelial metaplasia, hypertrophy of smooth muscle cells, and hyperproliferation (hyperplasia) of smooth muscle cells.

In a particular embodiment, the method and the medicament or pharmaceutical composition of the present invention can be used to prevent, alleviate and/or treat a moderate and/or severe asthma disease, COPD, systemic sclerosis or IPF accompanied by or comprising extracellular matrix protein production. In other words, the method and the medicament or pharmaceutical composition of the present invention can prevent, block or inhibit at least one symptom, feature or condition selected from hyperplasia of epithelial cells, epithelial metaplasia, hypertrophy of smooth muscle cells, hyperproliferation (hyperplasia) of smooth muscle cells, and extracellular matrix protein production dependent on TL1A-DR3 signal transduction, thereby inhibiting the exacerbation process of airway and/or lung tissue remodeling and inflammation in moderate and/or severe asthma, COPD, systemic sclerosis or IPF. Finally these can prevent a transition from chronic inflammation to tissue remodeling in airway and/or lung tissue.

In the present invention, the agent includes any agent that inhibits TL1A-DR3 interaction and/or TL1A-DR3 signal transduction. In one embodiment, the agent is at least one agent selected from a small molecule, an antibody, and a decoy molecule (such as a decoy-ligand and decoy-receptor).

Non-limiting examples of antibodies that may be used in the present invention include anti-TL1A antibody or anti-DR3 antibody which blocks TL1A-DR3 interaction and/or TL1A-DR3 signal transduction.

Non-limiting examples of decoy molecules that may be used in the present invention include TL1A or DR3 which is conjugated with a Fc region of immunoglobulin, or decoy receptor 3 (DcR3) and DcR3 which is conjugated with a Fc region of immunoglobulin.

A decoy receptor refers to a receptor that can bind to TL1A, to prevent, block or inhibit the interaction between TL1A and DR3 and/or TL1A-DR3 signal transduction. Non-limiting examples of a decoy receptor that may be used in the present invention include, but are not limited to, Decoy receptor 3 (DcR3).

A decoy ligand refers to a ligand that can bind to DR3, to prevent, block or inhibit the interaction between TL1A and DR3 and/or TL1A-DR3 signal transduction.

The agent inhibiting the TL1A-DR3 interaction may include, but is not limited to, the binding of a small molecule, antibody and/or a decoy molecule to either TL1A, or DR3 or both, preventing, reducing or blocking (inhibiting) the interaction between TL1A and DR3 receptor. Inhibition of TL1A-DR3 signal transduction may include, but is not limited to, the inhibition of function of any component involved in the TL1A-DR3 signal transduction pathway.

Small molecules refer to molecules of low molecular weight. Small molecules that inhibit TL1A-DR3 interaction include, but are not limited to, small molecules that can bind to TL1A, and/or DR3, or otherwise interfere with the interaction between TL1A and DR3 and/or TL1A-DR3 signal transduction.

In another embodiment, the method and the medicament or pharmaceutical composition of the present invention inhibits at least one symptom, feature or disease condition selected from migration of neutrophils, invasion of neutrophils, migration of eosinophils, invasion of eosinophils, hyperplasia of epithelial cells, epithelial metaplasia, hyperplasia of smooth muscle cells, hyperproliferation of smooth muscle cells, extracelluar matrix protein production and airway and/or lung tissue remodeling, by administering an agent which inhibits TL1A-DR3 interaction to a subject with a lung and/or airway disease, such as moderate and/or severe asthma disease, COPD, systemic sclerosis or IPF. Thus, the method and the medicament or pharmaceutical composition of the present invention, by inhibiting TL1A-DR3 interaction, TL1A-DR3 signal transduction, or a combination thereof, can inhibit exacerbation of airway and/or lung tissue remodeling, or can inhibit at least one symptom, feature or condition, in a subject with lung or airway disease such as moderate and/or severe asthma disease, COPD, IPF or systemic sclerosis.

In one embodiment of the present invention, antibodies used in the method or antibodies contained in the medicament or pharmaceutical composition of the present invention may include, but are not limited to, anti-DR3 monoclonal antibodies.

Preferably, the anti-DR3 antibodies comprise:

an antibody comprising amino acid sequences of CDR1 to CDR3 of VH of SEQ ID NOs:2-4 and amino acid sequences of CDR1 to CDR3 of VL of SEQ ID NOs:6-8;

an antibody comprising amino acid sequences of CDR1 to CDR3 of VH of SEQ ID NOs:10-12 and amino acid sequences of CDR1 to CDR3 of VL of SEQ ID NOs:14-16;

an antibody comprising amino acid sequences of CDR1 to CDR3 of VH of SEQ ID NOs:2, 3, and 21 and amino acid sequences of CDR1 to CDR3 of VL of SEQ ID NOs:6-8;

an antibody comprising amino acid sequences of CDR1 to CDR3 of VH of SEQ ID NOs:2, 3, and 21 and amino acid sequences of CDR1 to CDR3 of VL of SEQ ID NOs:6, 22, and 8;

an antibody comprising amino acid sequences of CDR1 to CDR3 of VH of SEQ ID NOs: 2, 3, and 21 and amino acid sequences of CDR1 to CDR3 of VL of SEQ ID NOs:6, 23, and 8;

an antibody comprising amino acid sequences of CDR1 to CDR3 of VH of SEQ ID NOs: 2, 3, and 21 and amino acid sequences of CDR1 to CDR3 of VL of SEQ ID NOs:6, 24, and 8;

an antibody comprising amino acid sequences of CDR1 to CDR3 of VH of SEQ ID NOs: 2-4 and amino acid sequences of CDR1 to CDR3 of VL of SEQ ID NOs: 6, 22, and 8;

an antibody comprising amino acid sequences of CDR1 to CDR3 of VH of SEQ ID NOs:2-4 and amino acid sequences of CDR1 to CDR3 of VL of SEQ ID NOs: 6, 23, and 8:

an antibody comprising amino acid sequences of CDR1 to CDR3 of VH of SEQ ID

NOs:2-4 and amino acid sequences of CDR1 to CDR3 of VL of SEQ ID NOs: 6, 24, and 8;
an antibody comprising an amino acid sequence of VH of SEQ ID NO:1 and an amino acid sequence of VL of SEQ ID NO:5,
an antibody comprising an amino acid sequence of VH of SEQ ID NO:9 and an amino acid sequence of VL of SEQ ID NO:13;
an antibody comprising an amino acid sequence of VH of SEQ ID NO:1 and an amino acid sequence of VL of SEQ ID NO:18;
an antibody comprising an amino acid sequence of VH of SEQ ID NO:1 and an amino acid sequence of VL of SEQ ID NO:19;
an antibody comprising an amino acid sequence of VH of SEQ ID NO:1 and an amino acid sequence of VL of SEQ ID NO:20;
an antibody comprising an amino acid sequence of VH of SEQ ID NO:17 and an amino acid sequence of VL of SEQ ID NO:18;
an antibody comprising an amino acid sequence of VH of SEQ ID NO:17 and an amino acid sequence of VL of SEQ ID NO:19; or
an antibody comprising an amino acid sequence of VH of SEQ ID NO:17 and an amino acid sequence of VL of SEQ ID NO:20.

In one embodiment of the present invention, antibodies used in a method or antibodies contained in a medicament or pharmaceutical composition of the present invention may include at least one anti-DR3 monoclonal antibody, wherein the anti-DR3 monoclonal antibody includes any antibody subclasses, preferably IgG subclass, more preferably IgG1, IgG2 or IgG4 subclass and their variants that are improved in stability, half-life and/or increased/decreased effector activity.

The at least one anti-DR3 monoclonal antibody may be selected from an anti-DR3 monoclonal antibody that includes, but is not limited to,

an anti-DR3 monoclonal antibody comprising an amino acid sequence having at least 90%, preferably at least 91%, 92%, 93%, 94%, more preferably at least 95%, 96%, 97%, 98% or 99% identity to each of the amino acid sequence of VH of SEQ ID NO:1 and VL of SEQ ID NO:5;

an anti-DR3 monoclonal antibody comprising an amino acid sequence having at least 90%, preferably at least 91%, 92%, 93%, 94%, more preferably at least 95%, 96%, 97%, 98% or 99% identity to each of the amino acid sequence of VH of SEQ ID NO:17 and VL of SEQ ID NO:18;

an anti-DR3 monoclonal antibody comprising an amino acid sequence having at least 90%, preferably at least 91%, 92%, 93%, 94%, more preferably at least 95%, 96%, 97%, 98% or 99% identity to each of the amino acid sequence of VH of SEQ ID NO:17 and VL of SEQ ID NO:19;

an anti-DR3 monoclonal antibody comprising an amino acid sequence having at least 90%, preferably at least 91%, 92%, 93%, 94%, more preferably at least 95%, 96%, 97%, 98% or 99% identity to each of the amino acid sequence of VH of SEQ ID NO:17 and VL of SEQ ID NO:20;

an anti-DR3 monoclonal antibody comprising an amino acid sequence having at least 90%, preferably at least 91%, 92%, 93%, 94%, more preferably at least 95%, 96%, 97%, 98% or 99% identity to each of the amino acid sequence of VH of SEQ ID NO:1 and VL of SEQ ID NO:18;

an anti-DR3 monoclonal antibody comprising an amino acid sequence having at least 90%, preferably at least 91%, 92%, 93%, 94%, more preferably at least 95%, 96%, 97%, 98% or 99% identity to each of the amino acid sequence of VH of SEQ ID NO:1 and VL of SEQ ID NO:19; or

an anti-DR3 monoclonal antibody comprising an amino acid sequence having at least 90%, preferably at least 91%, 92%, 93%, 94%, more preferably at least 95%, 96%, 97%, 98% or 99% identity to each of the amino acid sequence of VH of SEQ ID NO:1 and VL of SEQ ID NO:20.

The at least one anti-DR3 monoclonal antibody may be selected from an anti-DR3 monoclonal antibody that includes, but is not limited to,

an anti-DR3 monoclonal antibody comprising an amino acid sequence having at least 95%, 96%, 97%, 98% or 99% identity to each of the amino acid sequence of CDRs of VH of SEQ ID NOs:2-4 and each of the amino acid sequence of CDRs of VL of SEQ ID NOs:6-8,

an anti-DR3 monoclonal antibody comprising an amino acid sequence having at least 95%, 96%, 97%, 98% or 99% identity to each of the amino acid sequence of CDRs of VH of SEQ ID NOs:2, 3, 21 and each of the amino acid sequence of CDRs of VL of SEQ ID NOs:6-8,

an anti-DR3 monoclonal antibody comprising an amino acid sequence having at least 95%, 96%, 97%, 98% or 99% identity to each of the amino acid sequence of CDRs of VH of SEQ ID NOs:2, 3, 21 and each of the amino acid sequence of CDRs of VL of SEQ ID NOs:6, 22, 8,

an anti-DR3 monoclonal antibody comprising an amino acid sequence having at least 95%, 96%, 97%, 98% or 99% identity to each of the amino acid sequence of CDRs of VH of SEQ ID NOs:2, 3, 21 and each of the amino acid sequence of CDRs of VL of SEQ ID NOs:6, 23, 8,

an anti-DR3 monoclonal antibody comprising an amino acid sequence having at least 95%, 96%, 97%, 98% or 99% identity each of the amino acid sequence of CDRs of VH of SEQ ID NOs:2 and each of the amino acid sequence of CDRs 2, 3, 21 of VL of SEQ ID NOs:6, 24, 8,

an anti-DR3 monoclonal antibody comprising an amino acid sequence having at least 95%, 96%, 97%, 98% or 99% identity to each of the amino acid sequence of CDRs of VH of SEQ ID NOs:2-4 and each of the amino acid sequence of CDRs of VL of SEQ ID NOs: 6, 22, 8,

an anti-DR3 monoclonal antibody comprising an amino acid sequence having at least 95%, 96%, 97%, 98% or 99% identity to each of the amino acid sequence of CDRs of VH of SEQ ID NOs:2-4 and each of the amino acid sequence of CDRs of VL of SEQ ID NOs:6, 23, 8,

an anti-DR3 monoclonal antibody comprising an amino acid sequence having at least 95%, 96%, 97%, 98% or 99% identity to each of the amino acid sequence of CDRs of VH of SEQ ID NOs:2-4 and each of the amino acid sequence of CDRs of VL of SEQ ID NOs:6, 24, 8, or

an anti-DR3 monoclonal antibody comprising an amino acid sequence having at least 95%, 96%, 97%, 98% or 99% identity to each of the amino acid sequence of CDRs of VH of SEQ ID NOs:10-12 and each of the amino acid sequence of CDRs of VL of SEQ ID NOs:14-16.

Moreover antibodies used in the present method or antibodies contained in the medicament or pharmaceutical composition of the present invention may also include any affinity matured antibody clone being obtained from any kind screening method.

Having now fully described the invention, it will be appreciated by those skilled in the art that the same can be performed within a wide range of equivalent parameters, concentrations, and conditions without departing from the spirit and scope of the invention and without undue experimentation. While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and may be applied to the essential features hereinbefore set forth.

All references cited herein are hereby incorporated by reference in their entireties, whether previously specifically incorporated or not.

The following examples are provided to illustrate preferred aspects of the invention and are not intended to limit the scope of the invention.

### Example 1

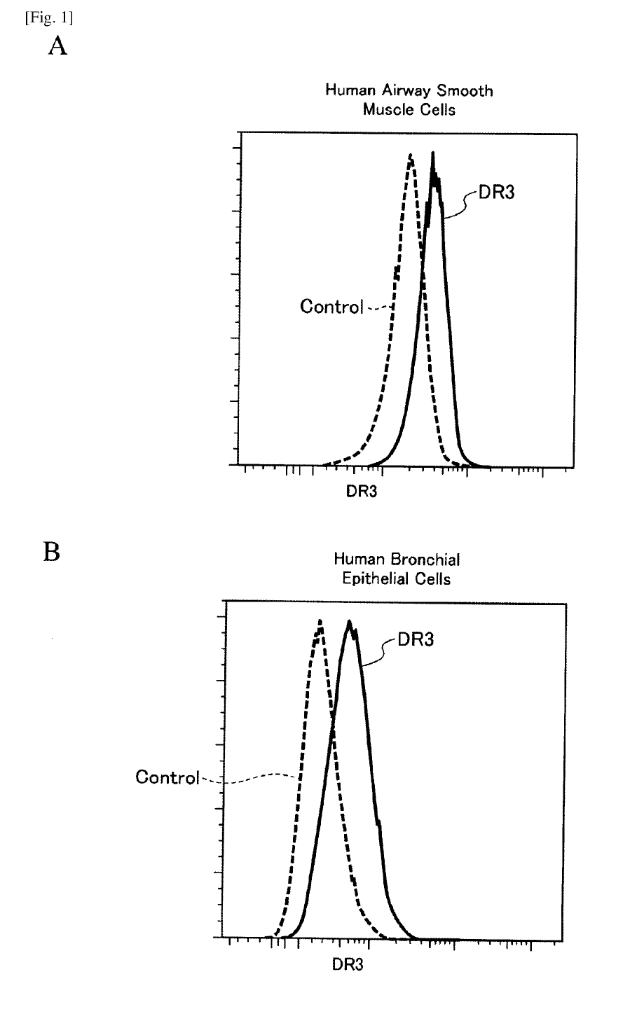

The receptor for TL1A is expressed on human lung structural cells linked to tissue remodeling. As shown in FIG. 1, primary normal human airway smooth muscle cells (FIG. 1A) (NHASM/BSMC), purchased from Lonza Group Ltd, and a human bronchial epithelium cell line (FIG. 1B) (BEAS2-B), were stained for DR3 with an anti-DR3 antibody. Isotype control is shown by dotted line. Data revealed that DR3 is expressed on structural cells that contribute to airway and/or lung tissue remodeling.

### Example 2

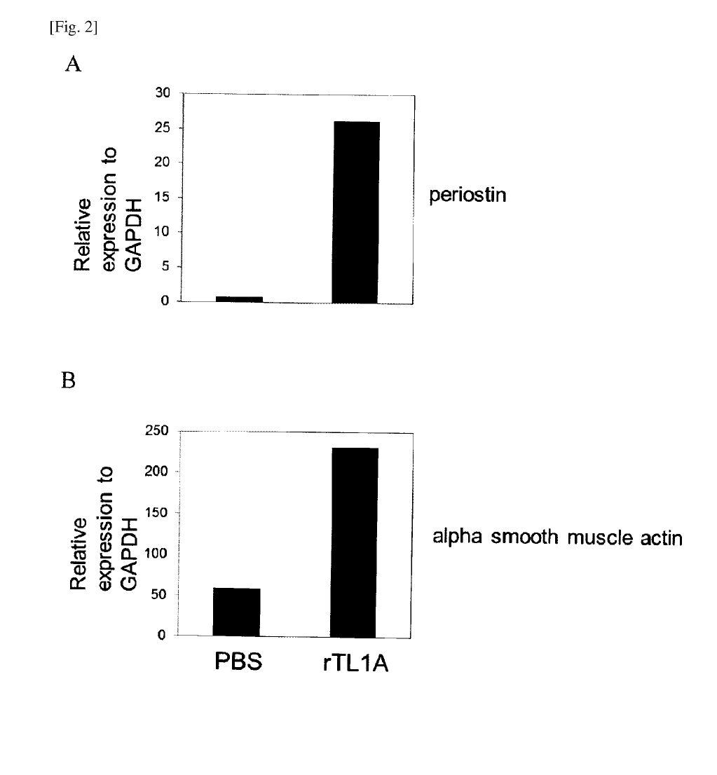

TL1A promoted tissue remodeling in human bronchial epithelial cells. As shown in FIG. 2, the human bronchial epithelium cell line BEAS-2B was stimulated with recombinant human TL1A (100 ng/ml) for 48 hrs and upregulation of periostin and alpha smooth muscle actin protein (by immunofluorescence, data not shown) and mRNA (by PCR, data relative expression to housekeeping gene) were assessed. The mRNA expression levels of periostin relative to GAPDH is shown in FIG. 2A, and the mRNA expression levels of alpha smooth muscle actin with respect to GAPDH is shown in FIG. 2B.

Immunofluorescence results were representative of 3 individual experiments, and mRNA data are from pooled cells from 3 individual cultures. Data revealed that TL1A can stimulate human bronchial epithelial cells to express the extracellular matrix protein periostin, and the structural protein alpha smooth muscle actin, which contribute directly to airway and/or lung tissue remodeling and decreased tissue function and elasticity.

### Example 3

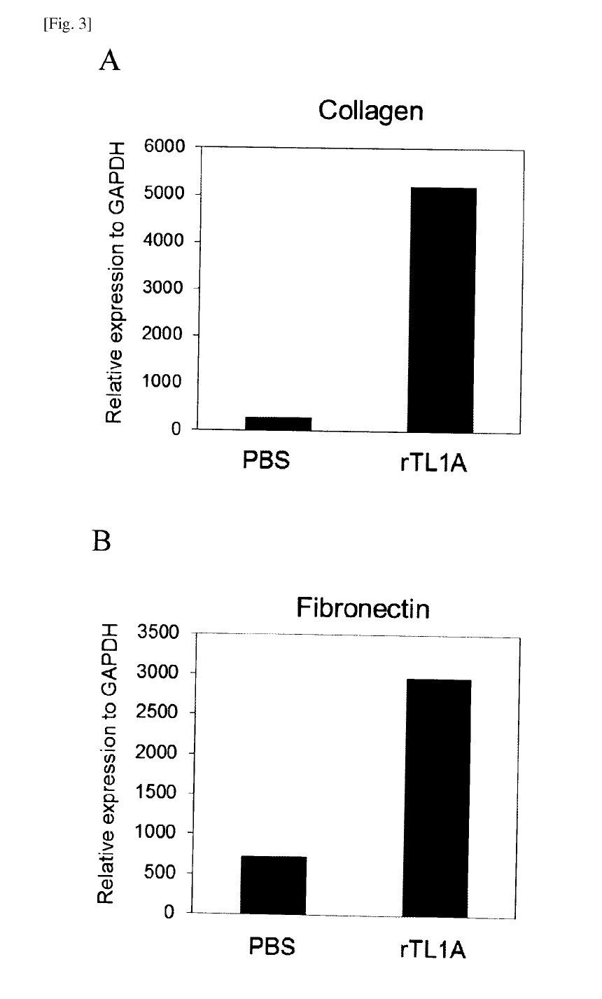

TL1A promoted tissue remodeling in airway smooth muscle cells. As shown in FIG. 3, primary normal human airway smooth muscle cells (NHASM/BSMC), purchased from Lonza Group Ltd, were stimulated with recombinant human TL1A (100 ng/ml) for 72 hrs and upregulation of collagen (Col1A1) and fibronectin mRNA were assessed (by PCR, data relative expression to housekeeping gene). mRNA data are from pooled cells from 3 individual cultures. Data revealed that TL1A can stimulate airway smooth muscle cells to express the extracellular matrix proteins collagen (FIG. 3A) and fibronectin (FIG. 3B) that contribute directly to airway and/or lung tissue remodeling and decreased tissue function and elasticity.

### Example 4

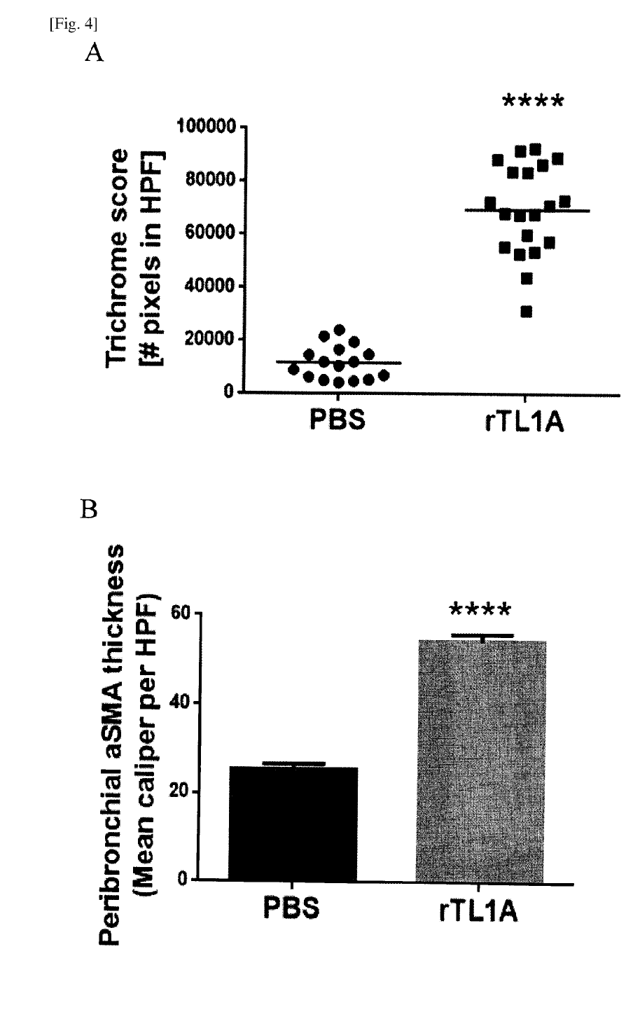

TL1A directly promoted lung tissue remodeling in wild-type mice. As shown in FIG. 4, unimmunized naive C57BL/6 mice were injected with PBS or 10 micrograms recombinant murine TL1A intratracheally on two consecutive days. On day 3, accumulation of collagen was analyzed in lung sections with trichrome blue immunohistochemical staining, and accumulation of smooth muscle around the bronchioles was assessed with immunofluorescence staining for alpha smooth muscle actin (aSMA) (data not shown). Quantitation of collagen deposition (area of trichrome staining) is shown in FIG. 4A and thickness of peribronchial smooth muscle is shown in FIG. 4B.

Results were representative of 5 individual mice or the average from 5 mice. ****p<0.001. Data revealed that recombinant TL1A in the absence of other stimuli can induce lung tissue remodeling in mice.

### Example 5

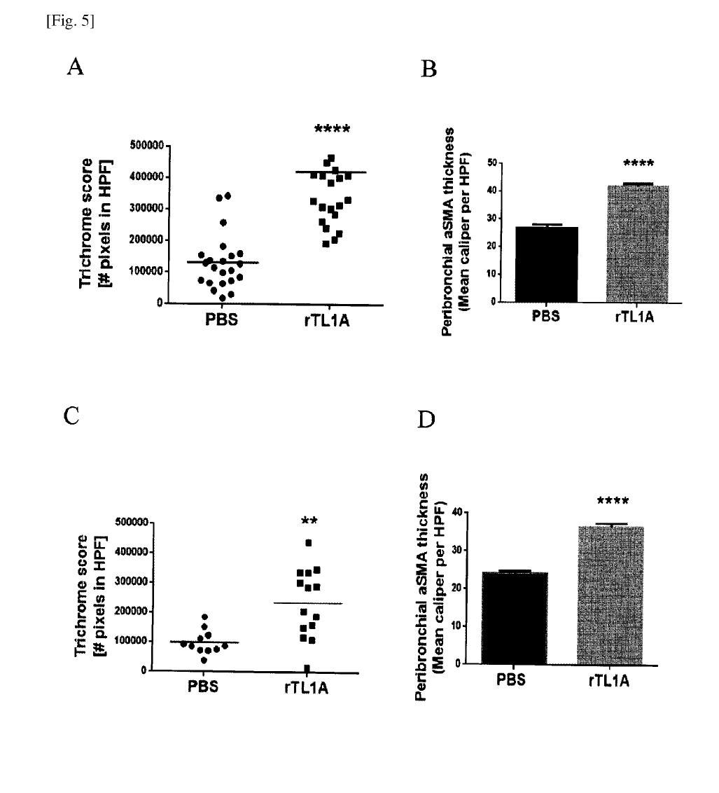

TL1A directly promoted lung tissue remodeling in mice lacking T cells and innate lymphoid cells. Unimmunized naive RAG−/− mice (FIGS. 5A and 5B) or RAG2gamma c−/− mice (FIGS. 5C and 5D) were injected with PBS or 10 micrograms recombinant murine TL1A intratracheally on two consecutive days. On day 3, accumulation of collagen was analyzed in lung sections with trichrome blue immunohistochemical staining, and accumulation of smooth muscle around the bronchioles was assessed with immunofluorescence for alpha smooth muscle actin (data not shown). Quantitation of collagen deposition (area of trichrome staining) (FIG. 5A) and thickness of peribronchial smooth muscle (FIG. 5B) in unimmunized naive RAG−/− mice, and quantitation of collagen deposition (area of trichrome staining) (FIG. 5C) and thickness of peribronchial smooth muscle (FIG. 5D) in RAG2gammac−/− mice are shown.

Results were representative of 5 individual mice or the average from 5 mice. ****p<0.001, **p<0.05. Data revealed that recombinant TL1A in the absence of other stimuli can induce lung tissue remodeling in mice lacking T cells (RAG−/−) and in mice lacking T cells and innate lymphoid cells (RAG2gammac−/−).

### Example 6

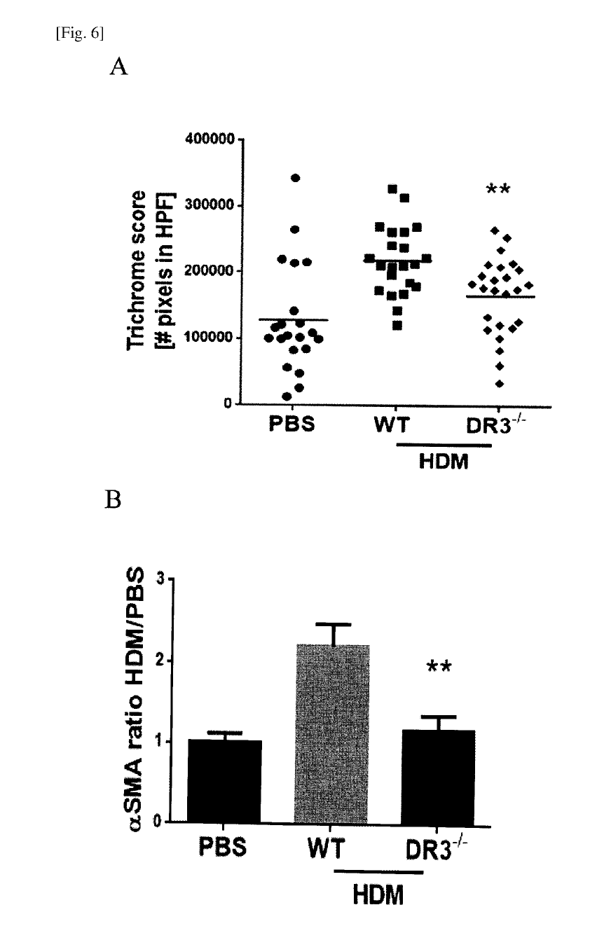

A genetic deficiency in DR3 reduced lung tissue remodeling driven by allergen. Wild-type or DR3−/− C57BL/6 mice were sensitized with house dust mite allergen extract (HDM) given intranasally on days 0, 7 and 14, to induce mild acute asthmatic lung inflammation. Mice were then treated chronically two times per week for another 4 weeks with intranasal HDM to promote lung tissue remodeling reminiscent of severe asthma. Lung sections were stained immunohistochemically with trichrome blue or with immunofluorescence for alpha smooth muscle actin (aSMA) (data not shown), and scored for accumulation of collagen (trichrome area) (FIG. 6A) and for change in peribronchial smooth muscle thickness compared to controls (FIG. 6B).

Results were representative of 5 individual mice or the average from 5 mice. ***p<0.005, **p<0.05. Data reveal that a DR3-deficiency protects mice from lung tissue remodeling in a model that displays disease features similar to those exhibited by patients with severe asthma.

### Example 7

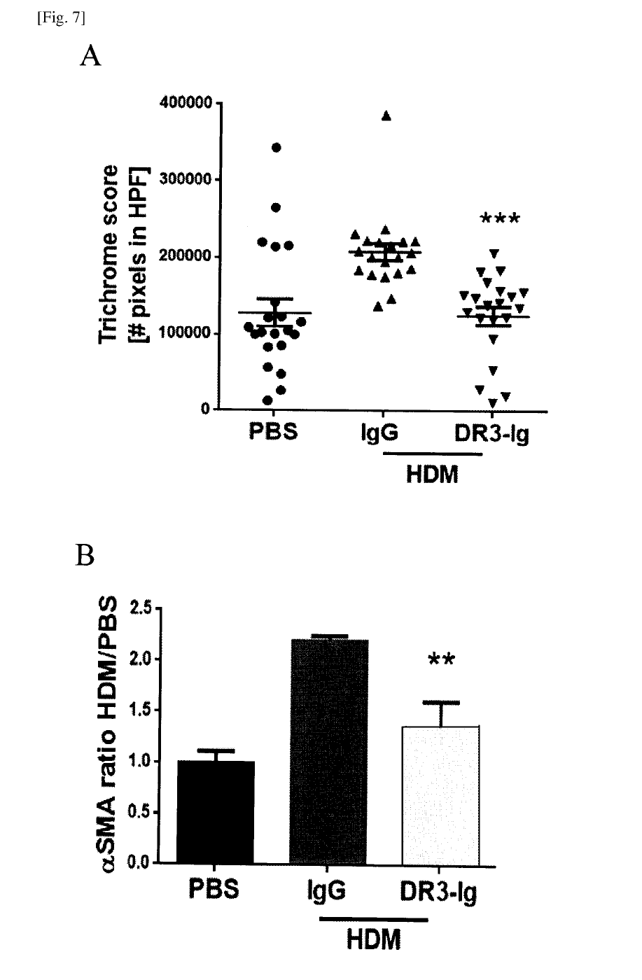

Therapeutic blocking of TL1A-DR3 interactions reduced lung tissue remodeling driven by allergen. Wild-type C57BL/6 mice were sensitized with house dust mite allergen extract (HDM) given intranasally on days 0, 7 and 14, to induce mild acute asthmatic lung inflammation. Mice were then treated chronically two times per week for another 4 weeks with intranasal HDM to promote lung tissue remodeling reminiscent of severe asthma. Control IgG, or an Fc fusion protein of DR3 (DR3-Ig) that binds TL1A and blocks it from interacting with DR3, were given therapeutically i.p. (100 micrograms) twice per week for the last 4 weeks. Lung sections were stained immunohistochemically with trichrome blue or with immunofluorescence for alpha smooth muscle actin (aSMA) (data not shown), and scored for accumulation of collagen (trichrome area) (FIG. 7A) and for change in peribronchial smooth muscle thickness compared to controls (FIG. 7B).

Results were representative of 5 individual mice or the average from 5 mice. ***p<0.005, **p<0.05. Data revealed that therapeutic inhibition of the interaction of TL1A with DR3 protects mice from lung tissue remodeling in a model that displays disease features similar to those exhibited by patients with severe asthma.

### Example 8

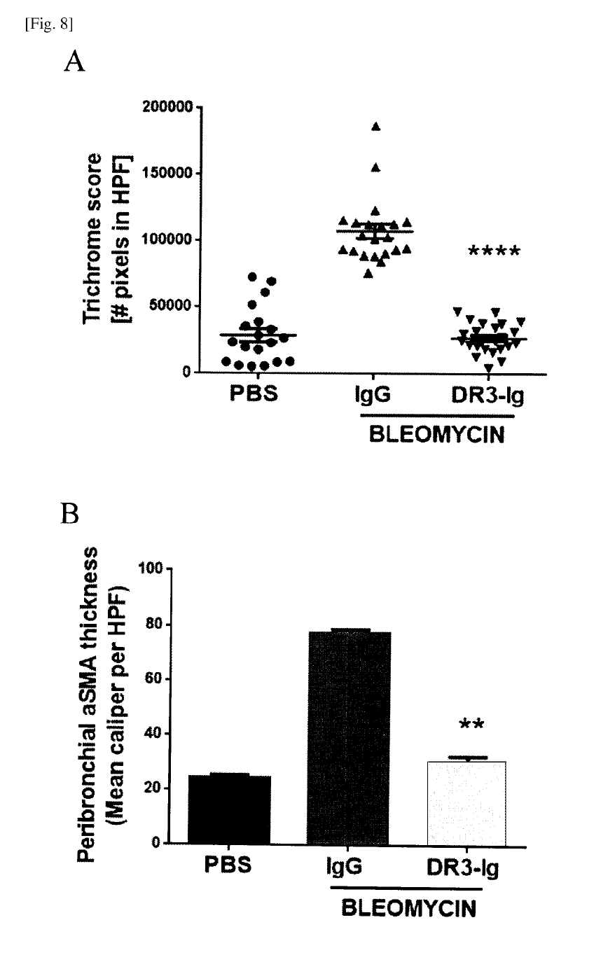

Blocking TL1A-DR3 interactions reduced lung tissue remodeling driven by bleomycin. Wild-type C57BL/6 mice were given the antibiotic bleomycin via intratracheal instillation. Bleomycin has been found to induce tissue remodeling in humans, and when injected into the lungs of mice promotes airway or lung remodeling. Control IgG, or an Fc fusion protein of DR3 (DR3-Ig) that binds TL1A and blocks it from interacting with DR3, were given i.p. (100 micrograms) at the time of treatment with bleomycin. After 7 days, lung sections were stained with trichrome blue or for alpha smooth muscle actin expression (aSMA) (data not shown), and scored for accumulation of collagen (trichrome area) (FIG. 8A) and for peribronchial smooth muscle thickness (FIG. 8B).

Results were representative of 10 individual mice or the average from 10 mice. ****p<0.001, **p<0.05. Data revealed that inhibition of the interaction of TL1A with DR3 protects mice from lung tissue remodeling in another model that displays disease features similar to those exhibited by patients with severe asthma. In addition, this mouse model is also known to display disease features similar to those exhibited by patients with IPF [Craing et al., J Immunol 2013; 190; 4283-4296] and systemic sclerosis [Yamamoto et al., Arch Dermatol Res (2006) 297; 333-344]. Therefore, data revealed that inhibition of the interaction of TL1A with DR3 protects mice from lung tissue remodeling in a model of relevance to IPF or systemic sclerosis.

### Example 9

Multiple inflammatory stimulants induced TL1A expression in neutrophils from healthy donors. mRNA levels were determined by Quantigene bDNA assay and normalized to the housekeeping gene PPIB.

(1) Isolation of Human Neutrohils from Whole Blood

Fresh heparinized whole blood was transferred into a conical tube. Blood (10 mL) was diluted with 0.9% NaCl (30 mL). Diluted whole blood was then underlayed with Ficoll (Amersham Biosciences cat.#17-1440-03). Pellets, which contain red blood cells and neutrophils were collected after centrifugation at 400×g for 40 min at room temperature with no brake. Pellets were resuspended in equal volume HBSS and 3% dextran solution (Spectrum cat.# DE130) and left undisturbed for 30 min to allow the red blood cells to sediment and a top clear layer containing the neutrophils to form. The cleared supernatant was transferred to a new tube and centrifuged 10 minutes at 250×g with the brake at room temperature. Pellets were resuspended in pre-chilled 0.2% NaCl (Fisher, cat.# S271-1) for 30 sec for a final hypotonic lysis of any remaining red blood cells and the isotonicity was restored by adding pre-chilled 1.6% NaCl. The neutrophils were centrifuged at 250×g for 6 minutes at 4° C. with the brake then resuspended in HBSS and kept on ice until used.

(2) Stimulation of Neutrophils

Neutrophils (2×10⁵ cells/well) were cultured with the following inflammatory stimuli in duplicate (Table 1) for 4 hrs at 37° C. Cells were collected after centrifugation and were lysed by adding 200 microL 1× Lysis mixture (Affymetrix, Cat. 12767) to each well, and the plates were incubated at 37° C. for 30 minutes for complete lysis. 80 microL of each lysate was transferred to a Quantigene 2.0 singleplex plate, and following the procedure specified by the manufacturer (Affymetrix, Quantigene 2.0 Reagent system user manual, Cat. 13074). The two mRNA probes used in this study were TL1A (Affymetrix, Cat. SA14783) and PPIB (Affymetrix, Cat. 50205).

Relative TL1A mRNA expression was calculated by dividing the TL1A probe relative light units (RLU) by the housekeeping gene PPIB probe RLU for each sample. The fold change in TL1A mRNA expression induced by each stimulant was further calculated by dividing the average relative expression level of TL1A in stimulated wells by the average RLU of duplicate unstimulated wells. Stimuli that induced at least a 5 fold increase in TL1A mRNA expression over unstimulated neutrophils are summarized in Table 2. Neutrophils from four healthy donors were tested with similar results.

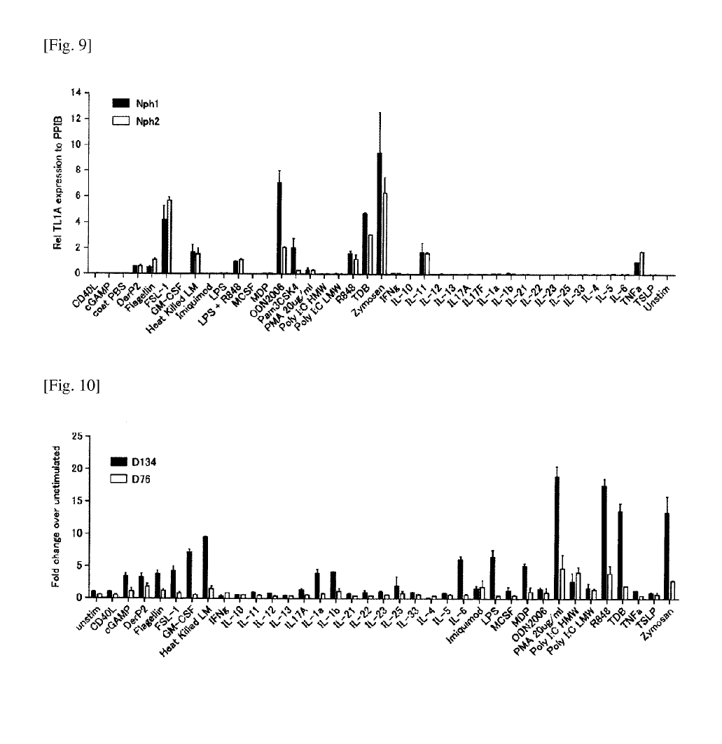

As shown in FIG. 9, the data revealed that TL1A expression in neutrophils was induced by various inflammatory stimuli implicated in moderate and/or severe asthma, IPF, or systemic sclerosis including TLR ligands, cytokines, and pathogen-associated molecular patterns (PAMPs), and identify a new source of TL1A, which can contribute directly to lung tissue remodeling and decreased tissue function and elasticity.

### Example 10

Multiple inflammatory stimulants induced TL1A expression in eosinophils from healthy donors. mRNA levels were determined by Quantigene bDNA assay and normalized to the housekeeping gene PPIB and fold change in TL1A expression over unstimulated cultures calculated.

(1) Isolation of Human Eosinophils from Whole Blood

Whole blood (10 mL) was mixed with 2 mL HetaSep solution (Stem Cell Technologies, Cat. 07906) by centrifugation at 50×g for 5 min with no brake. After incubation at room temperature for 7 minutes, the cleared supernatant containing the white blood cells was transferred to a new tube and washed with cold staining buffer (PBS+2% fetal bovine serum+1 mM EDTA) and centrifuged for 10 minutes at 500×g without the brake. The cell pellet was washed one more time with staining buffer and centrifuged at 120×g for 10 minutes with the brake. The cells were counted and resuspended at 5×10⁷ cells/mL and 50 microL/mL EasySep Negative Selection Human Eosinophil Enrichment Cocktail (Stem Cell Technologies, Cat. 19256) was added to the cells, mixed well, and incubated 10 minutes at 4° C. EasySep magnetic particles were added at 100 microL/mL, mixed well, and incubated at 4° C. for 10 minutes. The volume was increased to a total of 5 mL/10⁸ cells, mixed by pipetting, and the tube placed on the EasySep magnet for 10 minutes. The negatively enriched cells in the supernatant were collected by decanting the liquid into a new tube and a second 10 minute incubation on the magnet was performed to increase purity. After decanting the unbound cells (enriched eosinophils) to a new tube, the cells were washed with staining buffer at 300×g for 10 minutes, no brake and resuspended in 1 mL cold staining buffer for future use.

(2) Stimulation of Eosinophils

Eosinophils (2×10⁵ cells/well) were cultured with the following inflammatory stimuli in duplicate (Table 1) for 4 hrs at 37° C. Cells were collected after centrifugation and were lysed by adding 200 microL 1× Lysis mixture (Affymetrix, Cat. 12767) to each well, and the plates were incubated at 37° C. for 30 minutes for complete lysis. 80 microL of each lysate was transferred to a Quantigene 2.0 singleplex plate, and following the procedure specified by the manufacturer (Affymetrix, Quantigene 2.0 Reagent system user manual, Cat. 13074). The two mRNA probes used in this study were TL1A (Affymetrix, Cat. SA14783) and PPIB (Affymetrix, Cat. 50205).

Relative TL1A mRNA expression was calculated by dividing the TL1A probe relative light units (RLU) by the housekeeping gene PPIB probe RLU for each sample. The fold change in TL1A mRNA expression induced by each stimulant was further calculated by dividing the average relative expression level of TL1A in stimulated wells by the average RLU of duplicate unstimulated wells. Stimuli that induced at least a 2 fold increase in TL1A mRNA expression over unstimulated eosinophils in the two donors tested are summarized in Table 3.

As shown in FIG. 10, the data revealed that TL1A expression in eosinophils was induced by various inflammatory stimuli implicated in moderate and/or severe asthma, IPF or systemic sclerosis including TLR ligands and pathogen-associated molecular patterns (PAMPs), and identify a new source of TL1A, which can contribute directly to lung tissue remodeling and decreased tissue function and elasticity.

TABLE 1
Detailed description of stimulants or agonists for activation

| | | | Agonist Type or |
|---|---|---|---|
| Stimulant | Concentration | | Receptor |
| IL-1α | 50 | ng/mL | Cytokine |
| IL-1β | 50 | ng/mL | Cytokine |
| TNFα | 50 | ng/mL | Cytokine |
| IL-6 | 50 | ng/mL | Cytokine |
| IL-11 | 50 | ng/mL | Cytokine |
| IL-23 | 50 | ng/mL | Cytokine |
| IL-12 p70 | 50 | ng/mL | Cytokine |
| IL-10 | 50 | ng/mL | Cytokine |
| IL-17(A) | 50 | ng/mL | Cytokine |
| IL-21 | 50 | ng/mL | Cytokine |
| IL-22 | 50 | ng/mL | Cytokine |
| M-CSF | 50 | ng/mL | Cytokine |
| TSLP | 50 | ng/mL | Cytokine |
| IL-4 | 50 | ng/mL | Cytokine |
| IL-5 | 50 | ng/mL | Cytokine |
| IL-13 | 50 | ng/mL | Cytokine |
| IFNγ | 50 | ng/mL | Cytokine |
| GM-CSF | 50 | ng/mL | Cytokine |
| IL-33 | 50 | ng/mL | Cytokine |
| IL-25 = IL-17E | 50 | ng/mL | Cytokine |
| CD40 ligand | 50 | ng/mL | CD40 (co- |
| | | stimulator) | |
| MDP | 50 | ng/mL | NOD2 |
| cGAMP | 50 | ng/mL | STING |
| Zyomsan | 200000 | ng/mL | TLR2 and Dectin-1 |
| TDB | 20 | micrograms/mL | Mincle |
| Der P2 | 10000 | ng/mL | TLR4 |
| Heat killed LM | 1.00E+09 | cells/mL | TLR2 |
| Poly I:C HMW | 1 | micrograms/mL | TLR3 |
| Poly I:C LMW | 1 | micrograms/mL | TLR3 |
| LPS | 1 | micrograms/mL | TLR4 |
| Flagellin | 1 | micrograms/mL | TLR3 |
| FSL-1 | 0.1 | micrograms/mL | TLR2 and TLR6 |
| Imiquimod | 1 | micrograms/mL | TLR7 and TLR8 |
| ODN2006 | 5 | micromolar | TLR9 |
| R848 | 1 | micrograms/mL | TLR7 and TLR8 |
| LPS+ R848 | both from above | | TLR4 + TLR7 and |
| | | | TLR8 |
| PMA | 20 | micrograms/mL | Phorbol ester |

TABLE 2
Stimulants that induced at least a 5 fold increase in
TL1A expression by human neutrophils from 4 donors
Stimulant

PMA
LPS
R848
Zymosan
FSL-1
ODN2216
Imiquimod
Flagellin
Heat killed LM
DerP2
IL-1αL-12
TNF alpha
GM-CSF
TDB

TABLE 3
Stimulants that induced at least a 2 fold increase in
TL1A expression by human eosinophils from 2 donors
Stimulant

PMA
R848
Zymosan
Poly I:C HMW
TDB
DerP2

The present application claims benefit of U.S. Provisional Application Ser. No. 62/249,532 filed on Nov. 2, 2015; the entire contents of which are incorporated herein by reference.

### SEQUENCE LIST FREE TEXT

SEQ ID No.1: Description of the artificial sequence: 142A2-VH

SEQ ID No.2: Description of the artificial sequence: 142A2-HCDR1

SEQ ID No.3: Description of the artificial sequence: 142A2-HCDR2

SEQ ID No.4: Description of the artificial sequence: 142A2-HCDR3

SEQ ID No.5: Description of the artificial sequence: 142A2-VL

SEQ ID No.6: Description of the artificial sequence: 142A2-LCDR1

SEQ ID No.7: Description of the artificial sequence: 142A2-LCDR2

SEQ ID No.8: Description of the artificial sequence: 142A2-LCDR3

SEQ ID No.9: Description of the artificial sequence: 142S38BA-VH

SEQ ID No.10: Description of the artificial sequence: 142S38BA-HCDR1

SEQ ID No.11: Description of the artificial sequence: 142S38BA-HCDR2

SEQ ID No.12: Description of the artificial sequence: 142S38BA-HCDR3

SEQ ID No.13: Description of the artificial sequence: 142S38BA-VL

SEQ ID No.14: Description of the artificial sequence: 142S38BA-LCDR1

SEQ ID No.15: Description of the artificial sequence: 142S38BA-LCDR2

SEQ ID No.16: Description of the artificial sequence: 142S38BA-LCDR3

SEQ ID No.17: Description of the artificial sequence: 142A2-VH_G112R

SEQ ID No.18: Description of the artificial sequence: 142A2-VL_A51E

SEQ ID No.19: Description of the artificial sequence: 142A2-VL_L54Q

SEQ ID No.20: Description of the artificial sequence: 142A2-VL_A51E-L54Q

SEQ ID No.21: Description of the artificial sequence: 142A2-HCDR_G112R

SEQ ID No.22: Description of the artificial sequence: 142A2-LCDR2_A51E

SEQ ID No.23: Description of the artificial sequence: 142A2-LCDR2_L54Q

SEQ ID No.24: Description of the artificial sequence: 142A2-LCDR2_A51E/L54Q

1. A method of preventing, alleviating and/or treating an airway or lung disease comprising administering to a subject in need thereof, an effective amount of at least one agent which reduces or inhibits TL1A-DR3 interaction, TL1A-DR3 signal transduction or a combination thereof.

2. The method of claim 1, wherein the lung or airway disease involves infiltration of at least one cell selected from an eosinophil and neutrophil, and/or the lung disease or airway disease is selected from the group consisting of moderate and/or severe asthma, chronic obstructive pulmonary disease, idiopathic pulmonary fibrosis and systemic sclerosis.

3. (canceled)

4. The method of claim 1, wherein the subject has at least one symptom, feature or condition selected from hyperplasia of epithelial cells, epithelial metaplasia, hypertrophy of smooth muscle cells, hyperproliferation of smooth muscle cells, production of extracellular matrix, and airway or lung tissue remodeling associated with the lung disease or airway disease, which symptom, feature or condition is prevented, reduced, or inhibited by the inhibition of TL1A-DR3 interaction, TL1A-DR3 signal transduction or a combination thereof.

5. (canceled)

6. The method of claim 5, wherein the airway or lung tissue remodeling is characterized by at least one symptom, feature or condition selected from the group consisting of hyperplasia of epithelial cells, epithelial metaplasia, hypertrophy of smooth muscle cells, hyperproliferation of smooth muscle cells, and production of extracellular matrix.

7. The method of claim 1, wherein the at least one agent s selected from the group consisting of a small molecule, an antibody and a decoy molecule.

8. The method of claim 7, wherein the antibody is an anti-TL1A antibody or an anti-DR3 antibody, and the decoy molecule is selected from the group consisting of a decoy TL1A ligand, a decoy DR3 receptor, a decoy receptor 3 (DcR3), a TL1A conjugated with a Fc region of immunoglobulin, a DR3 conjugated with a Fc region of immunoglobulin, and a decoy receptor 3 (DcR3) conjugated with a Fc region of immunoglobulin.

9. (canceled)

10. (canceled)

11. The method of claim 8, wherein the anti-DR3 antibody is an anti-DR3 monoclonal antibody selected from the group consisting of:
an antibody comprising CDR1 to CDR3 of VH of SEQ ID NOs:2-4 and CDR1 to CDR3 of VL of SEQ ID NOs:6-8;
an antibody comprising CDR1 to CDR3 of VH of SEQ ID NOs:10-12 and CDR1 to CDR3 of VL of SEQ ID NOs:14-16;
an antibody comprising CDR1 to CDR3 of VH of SEQ ID NOs:2, 3, 21 and CDR1 to CDR3 of VL of SEQ ID NOs:6-8;
an antibody comprising CDR1 to CDR3 of VH of SEQ ID NOs:2, 3, 21 and CDR1 to CDR3 of VL of SEQ ID NOs:6, 22, 8;
an antibody comprising CDR1 to CDR3 of VH of SEQ ID NOs: 2, 3, 21 and CDR1 to CDR3 of VL of SEQ ID NOs:6, 23, 8;
an antibody comprising CDR1 to CDR3 of VH of SEQ ID NOs: 2, 3, 21 and CDR1 to CDR3 of VL of SEQ ID NOs:6, 24, 8;
an antibody comprising CDR1 to CDR3 of VH of SEQ ID NOs: 2-4 and CDR1 to CDR3 of VL of SEQ ID NOs: 6, 22, 8;
an antibody comprising CDR1 to CDR3 of VH of SEQ ID NOs:2-4 and CDR1 to CDR3 of VL of SEQ ID NOs: 6, 23, 8;
an antibody comprising CDR1 to CDR3 of VH of SEQ ID NOs:2-4 and CDR1 to CDR3 of VL of SEQ ID NOs: 6, 24, 8;
an antibody comprising VH of SEQ ID NO:1 and VL of SEQ ID NO:5;
an antibody comprising VH of SEQ ID NO:9 and VL of SEQ ID NO:13;
an antibody comprising VH of SEQ ID NO:1 and VL of SEQ ID NO:18;
an antibody comprising VH of SEQ ID NO:1 and VL of SEQ ID NO:19;
an antibody comprising VH of SEQ ID NO:1 and VL of SEQ ID NO:20;
an antibody comprising VH of SEQ ID NO:17 and VL of SEQ ID NO:18;
an antibody comprising VH of SEQ ID NO:17 and VL of SEQ ID NO:19; and
an antibody comprising VH of SEQ ID NO:17 and VL of SEQ ID NO:20.

12. A method of preventing, reducing, and/or inhibiting airway or lung tissue remodeling comprising administering to a subject with lung disease or airway disease, an effective amount of at least one agent which reduces or inhibits TL1A-DR3 interaction, TL1A-DR3 signal transduction or a combination thereof.

13. The method of claim 12, wherein airway or lung tissue remodeling is characterized by at least one symptom or condition selected from hyperplasia of epithelial cells, epithelial metaplasia, hypertrophy of smooth muscle cells, hyperproliferation of smooth muscle cells, production of extracellular matrix, and extracellular matrix protein production.

14. (canceled)

15. The method of claim 12, wherein the at least one agent is selected from a small molecule, an antibody, and a decoy molecule.

16. The method of claim 15, wherein the antibody is an anti-TL1A antibody or an anti-DR3 antibody, and the decoy molecule is selected from the group consisting of a decoy TL1A ligand, a decoy DR3 receptor, a decoy receptor 3 (DcR3) a TL1A conjugated with a Fc region of immunoglobulin, a DR3 conjugated with a Fc region of immunoglobulin, and a decoy receptor 3 (DcR3) conjugated with a Fc region of immunoglobulin.

17. (canceled)

18. (canceled)

19. The method of claim 16, wherein the anti-DR3 antibody is an anti-DR3 monoclonal antibody selected from the group consisting of:
an antibody comprising CDR1 to CDR3 of VH of SEQ ID NOs:2-4 and CDR1 to CDR3 of VL of SEQ ID NOs:6-8;
an antibody comprising CDR1 to CDR3 of VH of SEQ ID NOs:10-12 and CDR1 to CDR3 of VL of SEQ ID NOs:14-16;
an antibody comprising CDR1 to CDR3 of VH of SEQ ID NOs:2, 3, 21 and CDR1 to CDR3 of VL of SEQ ID NOs:6-8;
an antibody comprising CDR1 to CDR3 of VH of SEQ ID NOs:2, 3, 21 and CDR1 to CDR3 of VL of SEQ ID NOs:6, 22, 8;
an antibody comprising CDR1 to CDR3 of VH of SEQ ID NOs: 2, 3, 21 and CDR1 to CDR3 of VL of SEQ ID NOs:6, 23, 8;
an antibody comprising CDR1 to CDR3 of VH of SEQ ID NOs: 2, 3, 21 and CDR1 to CDR3 of VL of SEQ ID NOs:6, 24, 8;
an antibody comprising CDR1 to CDR3 of VH of SEQ ID NOs: 2-4 and CDR1 to CDR3 of VL of SEQ ID NOs: 6, 22, 8;
an antibody comprising CDR1 to CDR3 of VH of SEQ ID NOs:2-4 and CDR1 to CDR3 of VL of SEQ ID NOs: 6, 23, 8;
an antibody comprising CDR1 to CDR3 of VH of SEQ ID NOs:2-4 and CDR1 to CDR3 of VL of SEQ ID NOs: 6, 24, 8;
an antibody comprising VH of SEQ ID NO:1 and VL of SEQ ID NO:5;
an antibody comprising VH of SEQ ID NO:9 and VL of SEQ ID NO:13;
an antibody comprising VH of SEQ ID NO:1 and VL of SEQ ID NO:18;
an antibody comprising VH of SEQ ID NO:1 and VL of SEQ ID NO:19;
an antibody comprising VH of SEQ ID NO:1 and VL of SEQ ID NO:20;
an antibody comprising VH of SEQ ID NO:17 and VL of SEQ ID NO:18;
an antibody comprising VH of SEQ ID NO:17 and VL of SEQ ID NO:19; and
an antibody comprising VH of SEQ ID NO:17 and VL of SEQ ID NO:20.

20. A medicament for preventing, alleviating and/or treating a lung disease or airway disease comprising at least one agent which inhibits TL1A-DR3 interaction, TL1A-DR3 signal transduction or a combination thereof.

21. The medicament of any one of claim 20, wherein the at least one agent inhibits at least one symptom, feature or condition associated with the lung disease or airway disease selected from the group consisting of hyperplasia of epithelial cells, epithelial metaplasia, hypertrophy of smooth muscle cells, hyperproliferation of smooth muscle cells, production of extracellular matrix, and airway and/or lung tissue remodeling.

22. The medicament of claim 20, wherein the lung or airway disease is moderate and/or severe asthma, chronic obstructive pulmonary disease, idiopathic pulmonary fibrosis or systemic sclerosis.

23. The medicament of any one of claim 20, wherein the at least one agent is selected from the group consisting of a small molecule, an antibody and a decoy molecule.

24. The medicament of claim 23, wherein the antibody is an anti-TL1A antibody or an anti-DR3 antibody, and the decoy molecule is selected from the group consisting of a decoy TL1A ligand, a decoy DR3 receptor, a decoy receptor 3 (DcR3), a TL1A conjugated with a Fc region of immunoglobulin, a DR3 conjugated with a Fc region of immunoglobulin, and a decoy receptor 3 (DcR3) conjugated with a Fc region of immunoglobulin.

25. (canceled)

26. (canceled)

27. The medicament of claim 24, wherein the anti-DR3 antibody is an anti-DR3 monoclonal antibody selected from the group consisting of:
an antibody comprising CDR1 to CDR3 of VH of SEQ ID NOs:2-4 and CDR1 to CDR3 of VL of SEQ ID NOs:6-8;
an antibody comprising CDR1 to CDR3 of VH of SEQ ID NOs:10-12 and CDR1 to CDR3 of VL of SEQ ID NOs:14-16;
an antibody comprising CDR1 to CDR3 of VH of SEQ ID NOs:2, 3, 21 and CDR1 to CDR3 of VL of SEQ ID NOs:6-8;
an antibody comprising CDR1 to CDR3 of VH of SEQ ID NOs:2, 3, 21 and CDR1 to CDR3 of VL of SEQ ID NOs:6, 22, and 8;
an antibody comprising CDR1 to CDR3 of VH of SEQ ID NOs: 2, 3, 21 and CDR1 to CDR3 of VL of SEQ ID NOs:6, 23, and 8;
an antibody comprising CDR1 to CDR3 of VH of SEQ ID NOs: 2, 3, 21 and CDR1 to CDR3 of VL of SEQ ID NOs:6, 24, and 8;
an antibody comprising CDR1 to CDR3 of VH of SEQ ID NOs: 2-4 and CDR1 to CDR3 of VL of SEQ ID NOs: 6, 22, and 8;
an antibody comprising CDR1 to CDR3 of VH of SEQ ID NOs:2-4 and CDR1 to CDR3 of VL of SEQ ID NOs: 6, 23, and 8;
an antibody comprising CDR1 to CDR3 of VH of SEQ ID NOs:2-4 and CDR1 to CDR3 of VL of SEQ ID NOs: 6, 24, and 8;
an antibody comprising VH of SEQ ID NO:1 and VL of SEQ ID NO:5;
an antibody comprising VH of SEQ ID NO:9 and VL of SEQ ID NO:13;
an antibody comprising VH of SEQ ID NO:1 and VL of SEQ ID NO:18;
an antibody comprising VH of SEQ ID NO:1 and VL of SEQ ID NO:19;
an antibody comprising VH of SEQ ID NO:1 and VL of SEQ ID NO:20;
an antibody comprising VH of SEQ ID NO:17 and VL of SEQ ID NO:18;
an antibody comprising VH of SEQ ID NO:17 and VL of SEQ ID NO:19; and
an antibody comprising VH of SEQ ID NO:17 and VL of SEQ ID NO:20.

28. The method of claim 1, wherein the subject has an airway or lung disease.

29. The method of claim 28, wherein the at least one agent inhibits at least one symptom, feature or condition caused by the airway or lung disease selected from the group consisting of hyperplasia of epithelial cells, epithelial metaplasia, hypertrophy of smooth muscle cells, hyperproliferation of smooth muscle cells, production of extracellular matrix, and airway and/or lung tissue remodeling, and the airway or lung disease is moderate and/or severe asthma, chronic obstructive pulmonary disease, idiopathic pulmonary fibrosis or systemic sclerosis.

30. (canceled)

31. (canceled)

32. (canceled)

33. (canceled)

34. (canceled)

35. (canceled)


SEQUENCE LISTING (24 sequences)

SEQ ID NO: 1 - protein, 132 aa, Artificial
  Description of the artificial sequence 142A2-VH
QVQLQQSGPGLVKPSQTLSLTCAISGDSVSGYSAAWNWIRQSPSRGLEWLGRTYYRSKWYNDYAVSVKSRITINPDTSKNQFSLQLNSVTPEDTAVYYCARDYYGSESYYNGGYYYYGMDVWGQGTTVTVSS

SEQ ID NO: 2 - protein, 7 aa, Artificial
  Description of the artificial sequence 142A2-HCDR1
GYSAAWN

SEQ ID NO: 3 - protein, 18 aa, Artificial
  Description of the artificial sequence 142A2-HCDR2
RTYYRSKWYNDYAVSVKS

SEQ ID NO: 4 - protein, 20 aa, Artificial
  Description of the artificial sequence 142A2-HCDR3
DYYGSESYYNGGYYYYGMDV

SEQ ID NO: 5 - protein, 107 aa, Artificial
  Description of the artificial sequence 142A2-VL
AIQLTQSPSSLSASVGDRVTITCRASPGISSALAWYQQKPGKAPKLLMYDASSLESGVPSRFSGSGSGTDFTLTISSLQPEDFATYYCQQFNDYPLTFGGGTKVEIK

SEQ ID NO: 6 - protein, 11 aa, Artificial
  Description of the artificial sequence 142A2-LCDR1
RASPGISSALA

SEQ ID NO: 7 - protein, 7 aa, Artificial
  Description of the artificial sequence 142A2-LCDR2
DASSLES

SEQ ID NO: 8 - protein, 9 aa, Artificial
  Description of the artificial sequence 142A2-LCDR3
QQFNDYPLT

SEQ ID NO: 9 - protein, 117 aa, Artificial
  Description of the artificial sequence 142S38BA-VH
EVQLLESGGGLVQPGGSLRLSCAASGFTVSRYAMSWVRQAPGKGLEWVSAISDSGGRTYYADSVMGRFTISRDTSKNTLYLQMNSLRAEDTAVYFCAKEFGDYLDYWGQGTLVTVSS

SEQ ID NO: 10 - protein, 5 aa, Artificial
  Description of the artificial sequence 142S38BA-HCDR1
RYAMS

SEQ ID NO: 11 - protein, 17 aa, Artificial
  Description of the artificial sequence 142S38BA-HCDR2
AISDSGGRTYYADSVMG

SEQ ID NO: 12 - protein, 8 aa, Artificial
  Description of the artificial sequence 142S38BA-HCDR3
EFGDYLDY

SEQ ID NO: 13 - protein, 106 aa, Artificial
  Description of the artificial sequence 142S38BA-VL
EIVLTQSPATLSLSPGERATLSCRASQSVSSYLAWYQQKPGQAPRLLIYDASNRATGIPARFSGSGSGTDFTLTISSLEPEDFAVYYCQQRSNWWTFGQGTKVEIK

SEQ ID NO: 14 - protein, 11 aa, Artificial
  Description of the artificial sequence 142S38BA-LCDR1
RASQSVSSYLA

SEQ ID NO: 15 - protein, 7 aa, Artificial
  Description of the artificial sequence 142S38BA-LCDR2
DASNRAT

SEQ ID NO: 16 - protein, 8 aa, Artificial
  Description of the artificial sequence 142S38BA-LCDR3
QQRSNWWT

SEQ ID NO: 17 - protein, 132 aa, Artificial
  Description of the artificial sequence 142A2-VH_G112R
QVQLQQSGPGLVKPSQTLSLTCAISGDSVSGYSAAWNWIRQSPSRGLEWLGRTYYRSKWYNDYAVSVKSRITINPDTSKNQFSLQLNSVTPEDTAVYYCARDYYGSESYYNRGYYYYGMDVWGQGTTVTVSS

SEQ ID NO: 18 - protein, 107 aa, Artificial
  Description of the artificial sequence 142A2-VL_A51E
AIQLTQSPSSLSASVGDRVTITCRASPGISSALAWYQQKPGKAPKLLMYDESSLESGVPSRFSGSGSGTDFTLTISSLQPEDFATYYCQQFNDYPLTFGGGTKVEIK

SEQ ID NO: 19 - protein, 107 aa, Artificial
  Dscription of the artificial sequence 142A2-VL_ L54Q
AIQLTQSPSSLSASVGDRVTITCRASPGISSALAWYQQKPGKAPKLLMYDASSQESGVPSRFSGSGSGTDFTLTISSLQPEDFATYYCQQFNDYPLTFGGGTKVEIK

SEQ ID NO: 20 - protein, 107 aa, Artificial
  Description of the artificial sequence 142A2-VL_A51E-L54Q
AIQLTQSPSSLSASVGDRVTITCRASPGISSALAWYQQKPGKAPKLLMYDESSQESGVPSRFSGSGSGTDFTLTISSLQPEDFATYYCQQFNDYPLTFGGGTKVEIK

SEQ ID NO: 21 - protein, 20 aa, Artificial
  Description of the artificial sequence 142A2-HCDR_G112R
DYYGSESYYNRGYYYYGMDV

SEQ ID NO: 22 - protein, 7 aa, Artificial
  Description of the artificial sequence 142A2-LCDR2_A51E
DESSLES

SEQ ID NO: 23 - protein, 7 aa, Artificial
  Description of the artificial sequence 142A2-LCDR2_L54Q
DASSQES

SEQ ID NO: 24 - protein, 7 aa, Artificial
  Description of the artificial sequence 142A2-LCDR2_A51E/L54Q
DESSQES